(19) United States
(12) Patent Application Publication
(10) Pub. No.: US 2019/0039995 A1
(43) Pub. Date: Feb. 7, 2019

(54) METHOD FOR PREPARING SUBSTITUTED 4-AMINOINDANE DERIVATIVES

(71) Applicant: Bayer CropScience Aktiengesellschaft, Monheim am Rhein (DE)

(72) Inventors: Christoph SÄMANN, Düsseldorf (DE); Thomas HIMMLER, Odenthal (DE)

(21) Appl. No.: 16/074,639

(22) PCT Filed: Jan. 27, 2017
(86) PCT No.: PCT/EP2017/051778

§ 371 Date: Aug. 1, 2018

(30) Foreign Application Priority Data
Feb. 3, 2016 (EP) 16154083.6

(51) Int. Cl. C07C 209/68

(52) U.S. Cl. CPC C07C 209/68; C07C 211/60

(57) ABSTRACT

The present invention relates to a method for preparing substituted 4-aminoindane derivatives of the general formula (I) by cyclization,

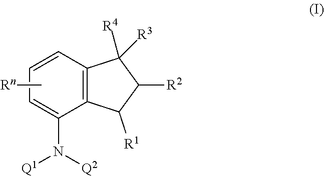

in which R, n, R¹, R², R³, R⁴, Q¹ and Q² have the definitions specified in the description.

The present invention relates to a method for preparing substituted aminoindane derivatives by cyclization.

4-Aminoindanes and corresponding derivatives are important intermediates for the preparation of bioactive compounds which can be used specifically for controlling harmful microorganisms in crop protection.

For instance, it is known that various pyrazole indanyl carboxamides have fungicidal activity (e.g. WO 1992/12970, WO 2012/065947, J. Org. Chem. 1995, 60, 1626 and WO 2012/084812).

It is also known that various pyridine indanyl carboxamides have fungicidal activity (e.g. EP-A 0256503, JP-A 1117864, J. Pesticide Sci. 1993, 18, 245).

In addition, it is known that some benzoyl indanyl amides have fungicidal activity (WO 2010/109301).

Chemical syntheses of 4-aminoindane derivatives have been described in the literature, but only allow the preparation of 4-aminoindanes with very limited substitution patterns (WO 2010/109301, WO 2014/103811, EP 0654464, U.S. Pat. No. 5,521,317). For instance, the methods described in WO 2010/109301 and in WO 2014/103811 only allow the synthesis of the 1,1,3-trimethyl-4-aminoindane derivative starting from aniline by condensation with acetone and exploit the rearrangement reaction described in EP 0654464 and U.S. Pat. No. 5,521,317.

A further possibility to prepare 4-aminoindane derivatives is described in WO 2013/167545 and WO 2013/167549. The synthesis is based on a Buchwald-Hartwig amination and thus enables a general synthetic route to substituted 4-aminoindanes. Disadvantages of this method are firstly the cost-intensive use of transition metal catalysts and secondly the problematic synthesis of the corresponding halo-substituted indane precursors. Furthermore, the amino function cannot be introduced directly by free NH₃, but rather requires the use of cost-intensive, protected ammonia derivatives.

Indanes without an amino function on the aromatic ring can be prepared by methods established in classical organic chemistry by Friedel-Crafts cyclizations. To this end, aromatic compounds having hydroxyalkyl or alkene side chains are converted to the corresponding indanes by addition of Brønsted acids such as HCl, HBr, HF, H₂SO₄, H₃PO₄, KHSO₄, AcOH, p-toluenesulphonic acid, polyphosphoric acid or of Lewis acids such as AlCl₃, BF₃, AgOTf. However, it has been shown that, with the exception of polyphosphoric acid, none of the reagents mentioned can be used to prepare 4-aminoindane derivatives by cyclization (J. S. Pizey (Ed.), “Synthetic Reagents 6” Wiley-VCH: New York 1985, 156-414). However, even the use of polyphosphoric acid is afflicted with some serious disadvantages. On the one hand, for example, the handling of the high-viscosity polyphosphoric acid is extremely inconvenient; on the other hand, an enormous amount of water is required to dissolve and dispose of this after completion of the reaction. In addition, a large amount of unwanted phosphate-containing waste is formed.

With regard to the disadvantages outlined above, there is a demand for a simplified method that can be carried out industrially and economically for the general preparation of substituted 4-aminoindane derivatives. The substituted 4-aminoindane derivatives obtainable by this desired method should preferably in this case be obtained in high yield and high purity. In particular, the desired method should enable the desired target compounds to be obtained without the need for complex purification methods such as column chromatography.

It has now been found, surprisingly, that 4-aminoindane derivatives can be prepared by a sulphonic acid-mediated cyclization reaction. Suitable sulphonic acids are preferably methanesulphonic acid or trifluoromethanesulphonic acid and particularly preferably trifluoromethanesulphonic acid. This is even more surprising since no such reaction has been described to date and those skilled in the art would have expected that exposure to these very strong acids would lead to decomposition of the starting material and/or the resulting products. Moreover, it had been assumed that—as in the use of other Brønsted or Lewis acids—successful cyclization would not take place.

Accordingly, the present invention relates to a novel method for preparing substituted 4-aminoindane derivatives of the general formula (I):

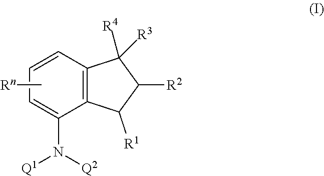

in which
R is mutually independently halogen, cyano, (C₁-C₁₂)alkyl, (C₃-C₇)cycloalkyl, (C₁-C₆)alkoxy, (C₁-C₆)alkylphenyl, aryl, cyano(C₁-C₆)alkyl, halo(C₁-C₆)alkyl having 1-9 identical or different halogen atoms, halo(C₃-C₇)cycloalkyl having 1-9 identical or different halogen atoms, halo(C₁-C₆)alkoxy having 1-9 identical or different halogen atoms, (C₁-C₆)alkoxycarbonyl(C₁-C₆)alkyl, (C₁-C₆)alkoxy(C₁-C₆)alkyl, (C₁-C₆)alkylsulphanyl, halo(C₁-C₆)alkylsulphanyl having 1-9 identical or different halogen atoms, (C₁-C₆)alkylsulphonyl or halo(C₁-C₆)alkylsulphonyl having 1-9 identical or different halogen atoms,
n is an integer from 0 to 3,
R¹, R², R³ and R⁴ are mutually independently hydrogen, (C₁-C₈)alkyl, (C₃-C₈)cycloalkyl, (C₃-C₈)cycloalkyl(C₁-C₈)alkyl, (C₃-C₈)cycloalkyl(C₃-C₈)cycloalkyl, (C₁-C₈)alkylphenyl, (C₁-C₈)alkoxy, aryl, cyano(C₁-C₈)alkyl, halo(C₁-C₈)alkyl having 1-9 identical or
   different halogen atoms, (C₁-C₈)alkoxycarbonyl(C₁-C₈)alkyl, (C₁-C₈)alkoxy(C₁-C₈)alkyl or halo(C₁-C₈)alkoxy(C₁-C₈)alkyl having 1-9 identical or different halogen atoms, and
Q¹ and Q² are mutually independently hydrogen, substituted (C₁-C₆)alkylsulphonyl, substituted alkoxycarbonyl(C₁-C₆)alkyl or substituted (C₁-C₆)haloalkylsulphonyl,
characterized in that alcohols of the general formulae (IIa), (IIb) or (IIc)

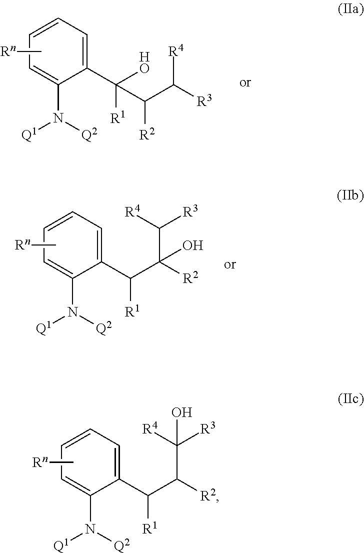

wherein the definitions of the residues R, n, Q¹, Q², R¹, R², R³ and R⁴ correspond to those of the general formula (I),
are reacted with sulphonic acids.

Preferred, particularly preferred and especially preferred definitions of the residues R, n, R¹, R², R³, R⁴, Q¹ and Q² listed in the formulae (I), (IIa), (IIb) and (IIc) mentioned above are elucidated below.

### Preferably

R is mutually independently halogen, (C₁-C₄)alkyl, (C₁-C₄)alkoxy, (C₁-C₄)alkylphenyl, aryl, cyano(C₁-C₄)alkyl, halo(C₁-C₄)alkyl having 1-9 identical or different halogen atoms, (C₁-C₄)alkoxycarbonyl(C₁-C₄)alkyl, (C₁-C₄)alkoxy(C₁-C₄)alkyl or halo(C₁-C₄)alkoxy(C₁-C₄)alkyl,
n is an integer from 0 to 3,
R¹, R², R³ and R⁴ are mutually independently hydrogen, (C₁-C₄)alkyl, (C₁-C₄)alkylphenyl, (C₁-C₄)alkoxy, aryl, cyano(C₁-C₄)alkyl, (C₁-C₄)alkoxycarbonyl(C₁-C₄)alkyl or (C₁-C₄)alkoxy(C₁-C₄)alkyl and
Q¹ and Q² are mutually independently hydrogen, substituted (C₁-C₄)alkylsulphonyl, substituted alkoxycarbonyl(C₁-C₄)alkyl or substituted (C₁-C₄)haloalkylsulphonyl.

### Particularly Preferably

R is mutually independently fluorine, chlorine, bromine, methyl or trifluoromethyl,
n is an integer from 0 to 1,
R¹, R², R³ and R⁴ are mutually independently hydrogen, methyl, ethyl, n-propyl, isopropyl, n-butyl, isobutyl, sec-butyl or tert-butyl and
Q¹ and Q² are mutually independently hydrogen, substituted (C₁-C₄)alkylsulphonyl, substituted alkoxycarbonyl(C₁-C₃)alkyl or substituted (C₁-C₃)haloalkylsulphonyl.

### Especially Preferably

n is 0 or
R is fluorine and n is 1, wherein fluorine is preferably in the 5-, 6- or 7-position, particularly preferably in the 6- or 7-position and especially preferably in the 7-position of the indane residue, or
R is trifluoromethyl and n is 1, wherein trifluoromethyl is preferably in the 5-, 6- or 7-position, particularly preferably in the 6- or 7-position and especially preferably in the 7-position of the indane residue,
R¹, R², R³ and R⁴ are mutually independently hydrogen, methyl, ethyl, n-propyl, n-butyl, isobutyl or sec-butyl and
Q¹ and Q² are hydrogen.

### More Especially Preferably

n is 0,
Q¹ and Q² are hydrogen,
wherein the definitions of the residues R¹, R², R³ and R⁴ correspond to the general, preferred, particularly preferred and especially preferred definitions listed for the formulae (I), (IIa), (IIb) and (IIc).

### More Especially Preferably

### R is F and n is 1,

Q¹ and Q² are hydrogen,
wherein the definitions of the residues R¹, R², R³ and R⁴ correspond to the general, preferred, particularly preferred and especially preferred definitions listed for the formulae (I), (IIa), (IIb) and (IIc).

### More Especially Preferably

### R is 7-F and n is 1,

Q¹ and Q² are hydrogen,
wherein the definitions of the residues R¹, R², R³ and R⁴ correspond to the general, preferred, particularly preferred and especially preferred definitions listed for the formulae (I), (IIa), (IIb) and (IIc).

### More Especially Preferably

### R is CF₃ and n is 1,

Q¹ and Q² are hydrogen,
wherein the definitions of the residues R¹, R², R³ and R⁴ correspond to the general, preferred, particularly preferred and especially preferred definitions listed for the formulae (I), (IIa), (IIb) and (IIc).

### More Especially Preferably

### R is 7-CF₃ and n is 1,

Q¹ and Q² are hydrogen,
wherein the definitions of the residues R¹, R², R³ and R⁴ correspond to the general, preferred, particularly preferred and especially preferred definitions listed for the formulae (I), (IIa), (IIb) and (IIc).

### General Definitions

In the definitions of the symbols given in the above formulae, collective terms which are generally representative of the following substituents were used:

Halogen: fluorine, chlorine, bromine and iodine and preferably fluorine, chlorine, bromine and more preferably fluorine, chlorine.

Alkyl: saturated, straight-chain or branched hydrocarbyl radicals having 1 to 12, preferably 1 to 6 and more preferably 1 to 3 carbon atoms, for example (but not limited to) C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. This definition also applies to alkyl as part of a composite substituent, for example cycloalkylalkyl, hydroxyalkyl etc., unless defined elsewhere like, for example, alkylthio, alkylsulphinyl, alkylsulphonyl, haloalkyl or haloalkylthio. When the alkyl is at the end of a composite substituent as in alkylcycloalkyl for example, the part of the composite substituent at the start, for example the cycloalkyl, may be mono- or polysubstituted identically or differently and independently by alkyl. The same also applies to composite substituents in which other radicals, for example alkenyl, alkynyl, hydroxyl, halogen, formyl etc., are at the end.

Alkoxy: saturated, straight-chain or branched alkoxy radicals having 1 to 6, preferably 1 to 3 carbon atoms, for example (but not limited to) C₁-C₆-alkoxy, such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy. This definition also applies to alkoxy as part of a composite substituent, for example haloalkoxy, alkynylalkoxy etc., unless defined elsewhere;

Cycloalkyl: monocyclic, saturated hydrocarbyl groups having 3 to 7, preferably 3 to 6 carbon ring members, for example (but not limited to) cyclopropyl, cyclopentyl and cyclohexyl. This definition also applies to cycloalkyl as part of a composite substituent, for example cycloalkylalkyl etc., unless defined elsewhere;

Haloalkyl: straight-chain or branched alkyl groups having 1 to 6, preferably 1 to 3 carbon atoms (as specified hereinabove), where some or all of the hydrogen atoms in these groups may be replaced by halogen atoms as specified hereinabove, for example (but not limited to) C₁-C₃-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2-difluoroethyl, 2,2-dichloro-2-fluoroethyl, 2,2,2-trichloroethyl, pentafluoroethyl and 1,1,1-trifluoroprop-2-yl. This definition also applies to haloalkyl as part of a composite substituent, for example haloalkylaminoalkyl etc., unless defined elsewhere;

Aryl groups in the context of the present invention, unless defined differently, are aromatic hydrocarbyl groups which may have zero, one, two or more heteroatoms (selected from O, N, P and S).

Specifically, this definition comprises, for example, the meanings cyclopentadienyl, phenyl, cycloheptatrienyl, cyclooctatetraenyl, naphthyl and anthracenyl; 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 1-pyrrolyl, 1-pyrazolyl, 1,2,4-triazol-1-yl, 1-imidazolyl, 1,2,3-triazol-1-yl, 1,3,4-triazol-1-yl; 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

Alkylaryl groups in the context of the present invention, unless defined differently, are aryl groups substituted by alkyl groups, which may have one alkyl chain and may have, in the aryl skeleton, zero, one or more heteroatoms (selected from O, N, P and S).

### Illustration of the Processes and Intermediates

4-Aminoindane derivatives of the general formula (I) may be prepared by the reaction according to the invention of the corresponding alcohols of the general formulae (IIa), (IIb) or (IIc) with sulphonic acids (see process (a)):

Process (a):

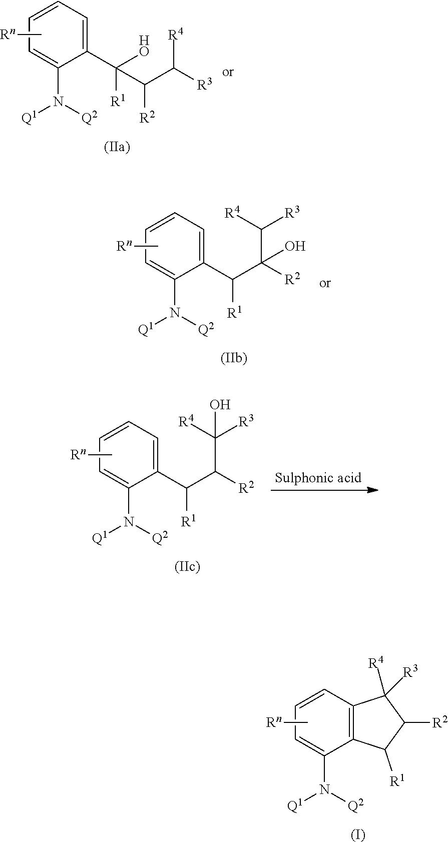

In the formulae (IIa), (IIb) and (IIc), the residues R, n, R¹, R², R³, R⁴, Q¹ and Q² are generally, preferably, particularly preferably and especially preferably the residues which have been defined above for the 4-aminoindanes of the general formula (I).

The compounds of the formulae (IIa), (IIb) or (IIc) used as starting materials may be prepared analogously to known methods (WO 2002/38542, WO 2006/120031).

Furthermore, compounds of the formulae (IIa) can also be prepared by the two-fold reaction of appropriately substituted aminobenzonitriles of the general formula (III) with Grignard reagents of the formulae (IVa) and (IVb) via the intermediately formed ketones of the formulae (Va) or (Vb).

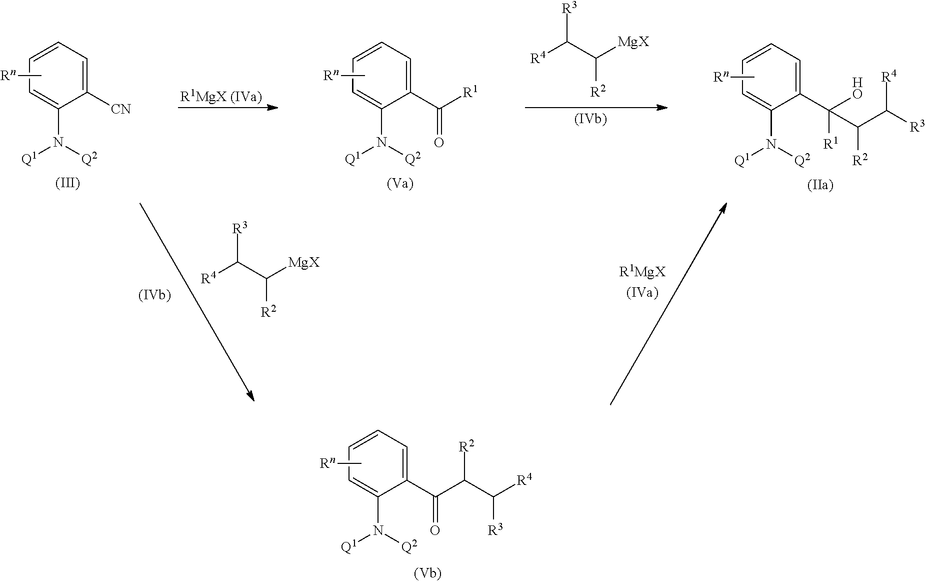

In the formulae (III), (IVa), (IVb), (Va) and (Vb), the residues R, n, R¹, R², R³, R⁴, Q¹ and Q² are generally, preferably, particularly preferably and especially preferably the residues which have been defined above for the 4-aminoindanes of the general formula (I).

In the formulae (IVa) and (IVb), X is preferably chlorine, bromine or iodine and particularly preferably chlorine or bromine.

The aminobenzonitriles of the formula (III) are known and in some cases commercially available.

The Grignard reagents of the formulae (IVa) and (IVb) are either commercially available or can be prepared from the corresponding chlorides, bromides or iodides by reaction with magnesium turnings by known literature methods.

A further process according to the invention for preparing 4-aminoindane derivatives of the general formula (I) is the reaction of the corresponding alkenes of the formulae (VIa) or (VIb) with sulphonic acids (see process (b)):

### Process (b):

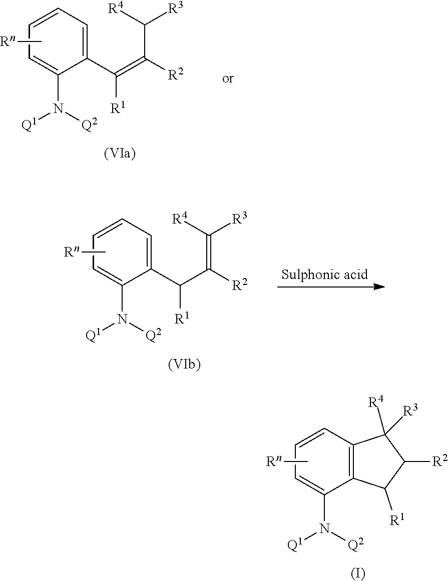

In the formulae (VIa) and (VIb), the residues R, n, R¹, R², R³, R⁴, Q¹ and Q² are generally, preferably, particularly preferably and especially preferably the residues which have been defined above for the aminoindanes of the general formula (I).

Compounds of formulae (VIa) or (VIb) may be prepared by standard methods of organic chemistry, for example by dehydration of the corresponding alcohols of formulae (IIa), (IIb) or (IIc).

4-Aminoindane derivatives of the general formula (I) may be prepared in a further process of the present invention by reacting the alkenes of the formulae (VIa′), (VIb′) or (VIc′) with sulphonic acids (see process (c)):

### Process (c):

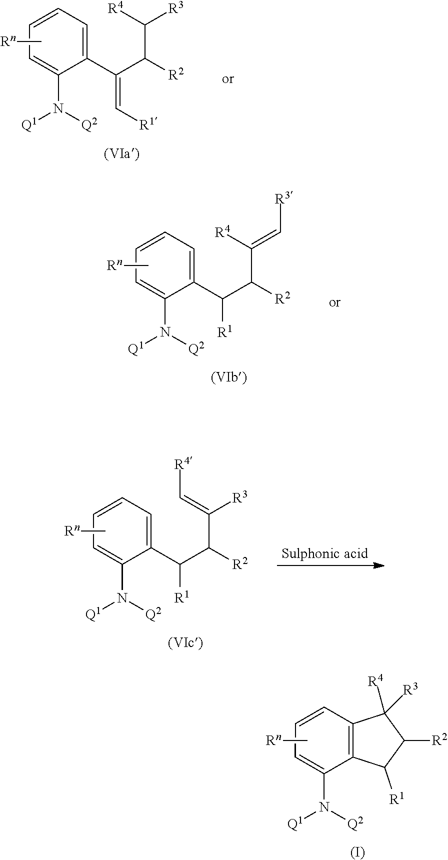

In the formulae (VIa′), (VIb′) and (VIc′), the residues R, n, R¹, R², R³, R⁴, Q¹ and Q² are generally, preferably, particularly preferably and especially preferably the residues which have been defined above for the aminoindanes of the general formula (I).

Compounds of the formulae (VIa′), (VIb′) or (VIc′) may be prepared by standard methods of organic chemistry, for example by dehydration of the corresponding alcohols of the formulae (IIa) or (IIc).

However, a prerequisite in this case is that the residues R¹ or R³/R⁴ permit formation of an alkene. The definition of the residues R^{1′}, R^{3′} and R^{4′} therefore derive appropriately from the definitions of R¹, R³ and R⁴.

R^{1′}, R^{3′} and R^{4′} are mutually independently (C₁-C₇)alkyl, (C₃-C₇)cycloalkyl,
(C₃-C₈)cycloalkyl(C₁-C₇)alkyl, (C₃-C₈)cycloalkyl(C₃-C₇)cycloalkyl, (C₁-C₇)alkylphenyl,
cyano(C₁-C₇)alkyl, halo(C₁-C₇)alkyl having 1-9 identical or different halogen atoms,
(C₁-C₈)alkoxycarbonyl(C₁-C₇)alkyl, (C₁-C₈)alkoxy(C₁-C₇)alkyl or halo(C₁-C₈)alkoxy(C₁-C₇)alkyl having 1-9 identical or different halogen atoms.

A further process of the present invention for preparing 4-aminoindane derivatives of the general formula (I) is the reaction of the corresponding tetrahydroquinolines of the general formula (VII) with sulphonic acids (see process (d)):

### Process (d):

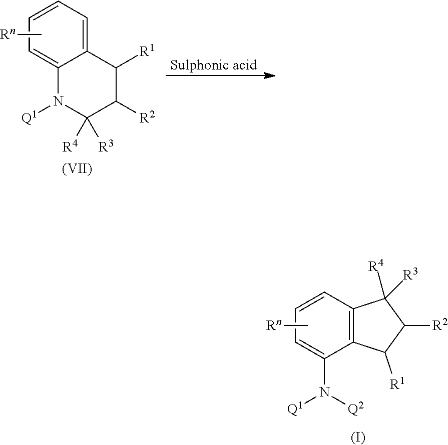

In the formula (VII), the residues R, n, R¹, R², R³, R⁴ and Q¹ are generally, preferably, particularly preferably and especially preferably the residues which have been defined above for the 4-aminoindanes of the general formula (I).

Compounds of the formula (VII) may be prepared analogously to known methods (WO 2010/109301, WO 2014/103811, EP 0654464, U.S. Pat. No. 5,521,317).

The method according to the invention for preparing substituted 4-aminoindane derivatives of the general formula (I) is based on different reaction steps, depending on the starting material, all of which can be mediated by the sulphonic acids. An overview of possible reaction steps, without being limited to these, is shown in scheme (I) (see below).

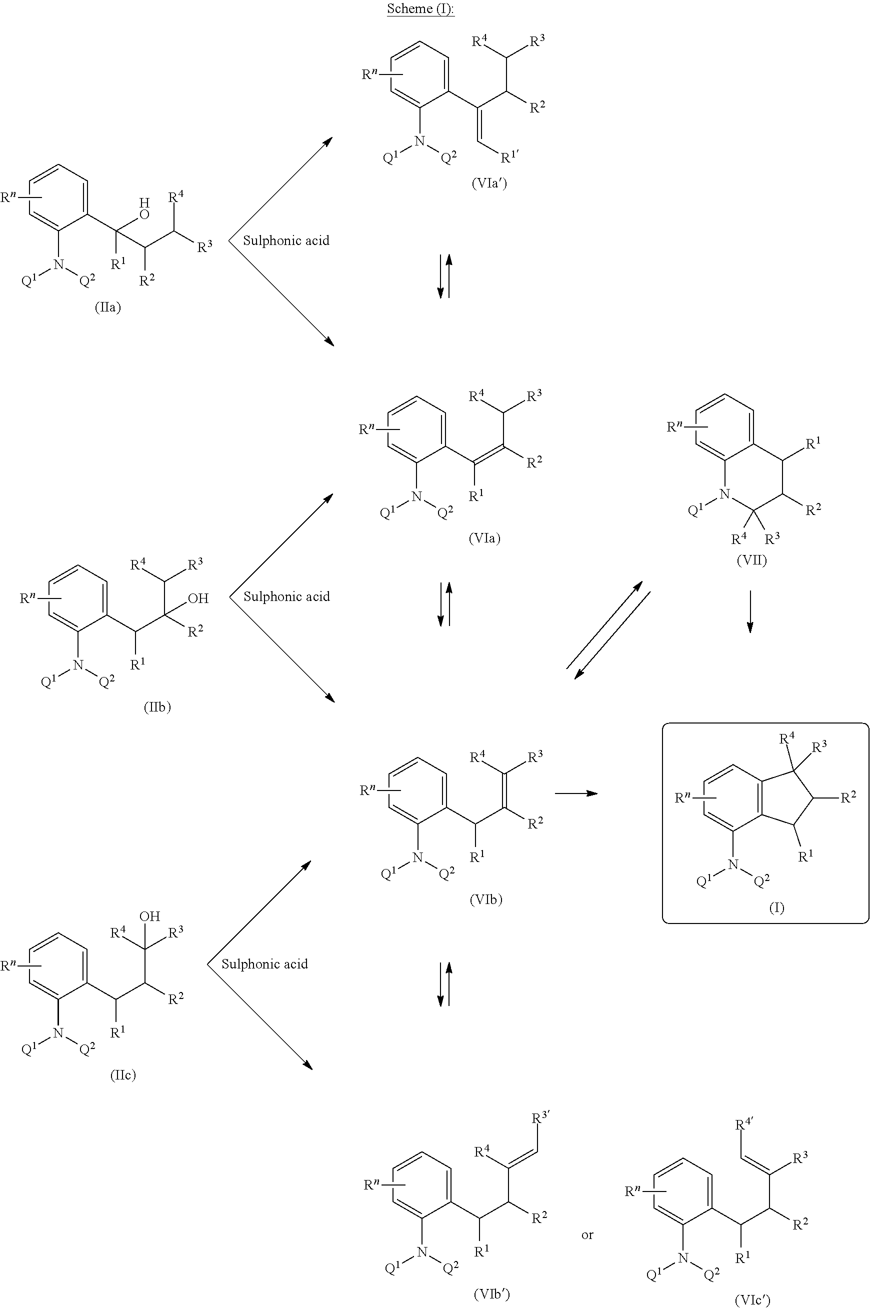

Starting from the alcohols of formulae (IIa), (IIb) or (IIc), reaction with the acid leads firstly, via the respective elimination, to the alkenes of formulae (VIa), (VIa′), (VIb) or (VIb′), which may interconvert by isomerization. Alkenes of the formula (VIb) can then either convert reversibly to the tetrahydroquinolines of the formula (VII) or cyclize irreversibly to the desired 4-aminoindane derivatives of the formula (I). All reaction steps, i.e. elimination, isomerization and cyclization are favoured or mediated by the sulphonic acids and ultimately lead to the desired substituted 4-aminoindanes of the general formula (I).

The processes (a), (b), (c) and (d) are preferably carried out with methanesulphonic acid or trifluoromethanesulphonic acid and particularly preferably with trifluoromethanesulphonic acid.

The processes (a), (b), (c) and (d), which employ trifluoromethanesulphonic acid, are preferably carried out in pure trifluoromethanesulphonic acid without solvent.

The processes (a), (b), (c) and (d), which employ methanesulphonic acid, are preferably carried out without solvent or in the following solvents: ethers such as tetrahydrofuran (THF), dioxane, diethyl ether, diglyme, methyl tert-butyl ether (MTBE), tert-amyl methyl ether (TAME), dimethyl ether, 2-methyl-THF; nitriles such as acetonitrile (ACN) or butyronitrile; ketones such as acetone, methyl isobutyl ketone (MIBK); aromatic hydrocarbons such as toluene, anisole, xylenes, mesitylene; esters such as ethyl acetate, isopropyl acetate, butyl acetate, pentyl acetate; alcohols such as methanol, ethanol, propanol, butanol, ethylene glycol; carbonates such as ethylene carbonate, propylene carbonate; amides such as N,N-dimethylacetamide (DMAc), N,N-dimethylformamide (DMF), N-methylpyrrolidone; halohydrocarbons and halogenated aromatic hydrocarbons, particularly chlorohydrocarbons such as tetrachloroethylene, tetrachloroethane, dichloropropane, methylene chloride (dichloromethane, DCM), dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, especially 1,2-dichlorobenzene, chlorotoluene, trichlorobenzene; fluorinated aliphatic and aromatic compounds such as trichlorotrifluoroethane, benzotrifluoride, 4-chlorobenzotrifluoride and water. It is also possible to use solvent mixtures.

Furthermore, processes (a), (b), (c) and (d), which employ methanesulphonic acid, are preferably carried out without solvent or in the following solvents: acetonitrile (ACN), butyronitrile, toluene, anisole, xylenes, mesitylene, N,N-dimethylacetamide (DMAc), N,N-dimethylformamide (DMF), N-methylpyrrolidone, halohydrocarbons and aromatic hydrocarbons, especially chlorohydrocarbons such as tetrachloroethylene, tetrachloroethane, dichloropropane, methylene chloride (dichloromethane, DCM), dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, especially 1,2-dichlorobenzene, chlorotoluene, trichlorobenzene; fluorinated aliphatic and aromatic compounds such as trichlorotrifluoroethane, benzotrifluoride, 4-chlorobenzotrifluoride and water. It is also possible to use solvent mixtures.

Furthermore, processes (a), (b), (c) and (d), which employ methanesulphonic acid, are particularly preferably carried out without solvent or in the following solvents: butyronitrile, toluene, xylenes, mesitylene, N,N-dimethylacetamide (DMAc), N,N-dimethylformamide (DMF), N-methylpyrrolidone, halohydrocarbons and aromatic hydrocarbons, especially chlorohydrocarbons such as tetrachloroethylene, tetrachloroethane, methylene chloride (dichloromethane, DCM), chloroform, carbon tetrachloride, 1,2-dichloroethane, chlorobenzene, bromobenzene, dichlorobenzene, especially 1,2-dichlorobenzene, chlorotoluene, trichlorobenzene and benzotrifluoride. It is also possible to use solvent mixtures.

Furthermore, processes (a), (b), (c) and (d), which employ methanesulphonic acid, are especially preferably carried out in pure methanesulphonic acid without solvent.

The amount of sulphonic acid which is used in processes (a), (b), (c) and (d) may be varied over a wide range but is preferably in the range of 0.1 to 100 equivalents, particularly preferably 0.1 to 50 equivalents and especially preferably 0.1 to 20 equivalents.

Processes (a), (b), (c) and (d) are generally conducted at standard pressure but may be carried out either under reduced pressure or at elevated pressure—generally between 0.1 and 100 bar.

Processes (a), (b), (c) and (d), which employ trifluoromethanesulphonic acid, are generally carried out at a temperature between −80° C. and 200° C., preferably between −20° C. and 140° C., especially preferably between −5° C. and 50° C.

Processes (a), (b), (c) and (d), which employ methanesulphonic acid, are generally carried out at a temperature between −80° C. and 250° C., preferably between 0° C. and 200° C., especially preferably between 0° C. and 150° C.

Depending on the type of substituents, the compounds of the general formula (I) can occur as geometric and/or optical isomers or as their corresponding isomeric mixtures in various compositions. These isomers are, for example, enantiomers, diastereomers or geometric isomers. As a consequence, the invention described here includes both the pure stereoisomers and every mixture of these isomers.

The desired compounds of the general formula (I) can be isolated and purified by diluting the reaction mixture with water with subsequent crystallization and release of the free 4-aminoindane derivative.

Such methods are known to those skilled in the art and particularly include the preferred crystallization from an organic solvent or a mixture of organic solvent and water.

The present invention is elucidated in detail by the examples which follow, although the examples should not be interpreted in such a manner that they restrict the invention.

### PREPARATION EXAMPLES

### Example 1: Synthesis of 1,1-dimethyl-3-propylindan-4-amine

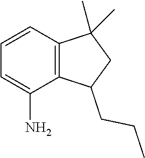

35.6 g (237 mmol) of trifluoromethanesulphonic acid are initially charged in a 250 mL reaction vessel and cooled to 0° C. Subsequently, 5.0 g (15.8 mmol, 70% purity) of 4-(2-aminophenyl)-2-methylheptan-4-ol are added and the reaction mixture is allowed to warm slowly to room temperature over 1 h. The reaction mixture is again cooled to 0° C. and 25 ml of water are added. The solid which precipitates is filtered off and washed three times with 10 ml of water. The solid is then recrystallized from toluene and the resulting solid is dried. This is then suspended in 50 mL of dichloromethane and 45% aqueous NaOH solution is added until a basic pH is reached. After stirring at room temperature for 30 min, the phases are separated and the aqueous phase is extracted twice with 30 mL of dichloromethane each time. The combined organic phases are washed once with 70 mL of saturated NaCl solution and dried over Na₂SO₄. The solvent is evaporated under reduced pressure and 2.4 g of 1,1-dimethyl-3-propylindan-4-amine (95% yield, 94.0% purity by HPLC) are obtained as a brown oil.

¹H NMR (CDCl₃) □ (ppm)=7.0 (t, 1H), 6.6 (d, 1H), 6.5 (d, 1H), 3.6 (br s, 2H), 3.05-3.1 (m, 1H), 2.1 (dd, 1H), 1.85-1.9 (m, 1H), 1.8 (dd, 1H), 1.5-1.55 (m, 1H), 1.35-1.4 (m, 2H), 1.3 (s, 3H), 1.2 (s, 3H), 1.0 (t, 3H).

### Comparative Example 1: Synthesis of 1,1-dimethyl-3-propylindan-4-amine

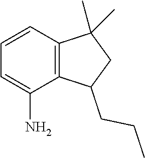

153.4 g (791 mmol) of polyphosphoric acid are initially charged in a 1000 mL reaction vessel, 18.5 g (79.0 mmol, 95% purity) of 4-(2-aminophenyl)-2-methylheptan-4-ol are added and the mixture is stirred overnight at 190° C. The reaction mixture is cooled and 300 ml of water are added over 1 h. The reaction mixture is then added dropwise to 200 mL of a cooled 45% aqueous NaOH solution. After stirring at room temperature for 30 min, 300 mL of dichloromethane are added and the mixture is stirred for a further 1.5 h. The phases are separated and the aqueous phase is extracted twice with 200 ml of dichloromethane each time. The combined organic phases are washed once with 500 mL of saturated NaCl solution, dried over Na₂SO₄ and the solvent is evaporated under reduced pressure. The crude product thus obtained is purified firstly by Kugelrohr distillation (100-120° C., 0.4 mbar) and subsequently by column chromatography (cyclohexane:ethyl acetate=9:1). 4.2 g of 1,1-dimethyl-3-propylindan-4-amine (25% yield, 95.8% purity by HPLC) are obtained as a brown oil.

### Example 2: Synthesis of 1,1-dimethyl-3-ethylindan-4-amine

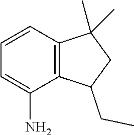

35.6 g (237 mmol) of trifluoromethanesulphonic acid are initially charged in a 250 mL reaction vessel and cooled to 0° C. Subsequently, 5.0 g (15.8 mmol, 65% purity) of 4-(2-aminophenyl)-2-methylhexan-4-ol are added and the reaction mixture is allowed to warm slowly to room temperature over 1 h. The reaction mixture is again cooled to 0° C. and 25 ml of water are added. The solid which precipitates is filtered off and washed three times with 10 ml of water. The solid is then recrystallized from toluene and the resulting solid is dried. This is then suspended in 50 mL of dichloromethane and 45% aqueous NaOH solution is added until a basic pH is reached. After stirring at room temperature for 30 min, the phases are separated and the aqueous phase is extracted twice with 30 mL of dichloromethane each time. The combined organic phases are washed once with 70 mL of saturated NaCl solution and dried over Na₂SO₄. The solvent is evaporated under reduced pressure and 2.5 g of 1,1-dimethyl-3-ethylindan-4-amine (84% yield, 92.8% purity by HPLC) are obtained as a brown oil.

¹H NMR (CDCl₃) □ (ppm)=7.0 (t, 1H), 6.6 (d, 1H), 6.5 (d, 1H), 3.6 (br s, 2H), 3.0-3.1 (m, 1H), 2.1 (dd, 1H), 1.9-2.0 (m, 1H), 1.8 (dd, 1H), 1.4-1.5 (m, 1H), 1.3 (s, 3H), 1.2 (s, 3H), 1.0 (t, 3H).

### Example 3: Synthesis of 1,1-dimethyl-3-ethylindan-4-amine

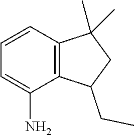

14.0 g (145 mmol) of methanesulphonic acid are initially charged in a 100 mL reaction vessel, 2.5 g (10.0 mmol, 80% purity) of 4-(2-aminophenyl)-2-methylhexan-4-ol are added and the reaction mixture is stirred at 120° C. for 2 h. The reaction mixture is cooled to 0° C. and 45% aqueous NaOH solution is added until a basic pH is reached. After stirring at room temperature for 30 min, the phases are separated and the aqueous phase is extracted twice with 30 mL of dichloromethane each time. The combined organic phases are washed once with 50 mL of saturated NaCl solution and dried over Na₂SO₄. The solvent is evaporated under reduced pressure and 3.6 g of 1,1-dimethyl-3-ethylindan-4-amine (52% yield by HPLC) are obtained as crude product.

### Comparative Example 2: Synthesis of 1,1-dimethyl-3-ethylindan-4-amine

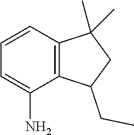

81.4 g (420 mmol) of polyphosphoric acid are initially charged in a 500 mL reaction vessel, 9.4 g (42.0 mmol, 93% purity) of 4-(2-aminophenyl)-2-methylhexan-4-ol are added and the mixture is stirred overnight at 190° C. The reaction mixture is cooled and 140 ml of water are added over 1 h. The reaction mixture is then added dropwise to 100 mL of a cooled 45% aqueous NaOH solution. After stirring at room temperature for 30 min, 150 mL of dichloromethane are added and the mixture is stirred for a further 1.5 h. The phases are separated and the aqueous phase is extracted twice with 100 ml of dichloromethane each time. The combined organic phases are washed once with 250 mL of saturated NaCl solution, dried over Na₂SO₄ and the solvent is evaporated under reduced pressure. The crude product thus obtained is purified by Kugelrohr distillation (85-105° C., 0.4 mbar). 1.8 g of 1,1-dimethyl-3-ethylindan-4-amine (22% yield, 95.6% purity by HPLC) are obtained as a brown oil.

The cyclizations described above were carried out using trifluoromethanesulphonic acid, methanesulphonic acid or polyphosphoric acid. As is evident from Table 1, cyclization experiments with other Brønsted or Lewis acids were unsuccessful.

TABLE 1
cyclization experiments of 4-(2-aminophenyl)-2-methylhexan-4-ol to give
1,1-dimethyl-3-ethylindan-4-amine using various Brønsted or Lewis acids:

| | Brønsted or | | |
|---|---|---|---|
| Input | Lewis acid | Solvent | Yield |
| 1 | TfOH | — | 84% |
| 2 | MsOH | — | 52%* |
| 3 | Polyphosphoric | — | 22% |
| | acid | | |
| 4 | Diphosphoric acid | — | 0% |
| 5 | Trifluoroacetic acid | — | 0% |
| 6 | HCl | Dioxane | 0% |
| 7 | HCl | Water | 0% |
| 8 | H₂SO₄ | — | 0% |
| 9 | p-TsOH | — | 0% |
| 10 | AlCl₃ | Xylene | 0% |
| 11 | BF₃•OEt₂ | — | 0% |
| 12 | BF₃•OEt₂ | Xylene | 0% |

*yield by HPLC

### Example 4: Synthesis of 4-(2-aminophenyl)-2-methylheptan-4-ol

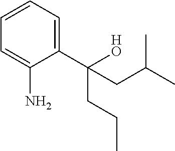 [SMILES: CCCC(O)(CC(C)C)c1ccccc1N]

160 mL (318 mmol, 2M in THF) of n-propylmagnesium chloride solution are initially charged in a baked-out 2 L reaction vessel under argon and the mixture is cooled to 0° C. 15 g (127 mmol) of 2-aminobenzonitrile are dissolved in 150 ml of dry THF and added dropwise at 0° C. over 1 h. After addition has ended, the mixture is stirred at room temperature for 30 min. 320 mL of aqueous HCl (1M) are initially charged in a further reaction vessel and cooled to 0° C. The reaction mixture is slowly added dropwise and subsequently adjusted to pH 4 with concentrated HCl. The phases are separated and the aqueous phase is extracted twice with 200 ml of ethyl acetate each time. The combined organic phases are washed once with 400 mL of saturated NaCl solution, dried over Na₂SO₄ and the solvent is evaporated under reduced pressure.

The ketone thus obtained is dissolved in 130 mL of dry THF and added dropwise under argon to 100 mL (200 mmol, 2M in THF) of isobutylmagnesium chloride at 0° C. over 1 h. After addition has ended, the mixture is stirred at room temperature for 30 min. 200 mL of aqueous HCl (1M) are initially charged in a further reaction vessel and cooled to 0° C. The reaction mixture is slowly added dropwise and subsequently adjusted to pH 4 with concentrated HCl. The phases are separated and the aqueous phase is extracted twice with 200 ml of ethyl acetate each time. The combined organic phases are washed once with 400 mL of saturated NaCl solution, dried over Na₂SO₄ and the solvent is evaporated under reduced pressure. 18.9 g (65% yield over two stages, 94.6% purity by HPLC) of 4-(2-aminophenyl)-2-methylheptan-4-ol are obtained as a yellow oil.

¹H NMR (CD₃CN) □ (ppm)=7.0 (d, 1H), 6.9 (t, 1H), 6.5-6.6 (m, 2H), 4.9 (br s, 2H), 2.9 (s, 1H), 1.8-1.9 (m, 2H), 1.8 (d, 2H), 1.6 (sept, 1H), 1.3-1.4 (m, 2H), 0.9 (t, 3H), 0.9 (d, 3H), 0.8 (d, 3H).

1. Method for preparing substituted 4-aminoindane derivative of formula (I) 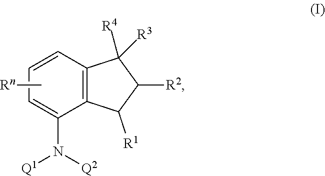
in which
R is mutually independently halogen, cyano, (C₁-C₁₂)alkyl, (C₃-C₇)cycloalkyl, (C₁-C₆)alkoxy, (C₁-C₆)alkylphenyl, aryl, cyano(C₁-C₆)alkyl, halo(C₁-C₆)alkyl having 1-9 identical or different halogen atoms, halo(C₃-C₇)cycloalkyl having 1-9 identical or different halogen atoms, halo(C₁-C₆)alkoxy having 1-9 identical or different halogen atoms, (C₁-C₆)alkoxycarbonyl(C₁-C₆)alkyl, (C₁-C₆)alkoxy(C₁-C₆)alkyl, (C₁-C₆)alkylsulphanyl, halo(C₁-C₆)alkylsulphanyl having 1-9 identical or different halogen atoms, (C₁-C₆)alkylsulphonyl or halo(C₁-C₆)alkylsulphonyl having 1-9 identical or different halogen atoms,
n is an integer from 0 to 3,
R¹, R², R³ and R⁴ are mutually independently hydrogen, (C₁-C₈)alkyl, (C₃-C₈)cycloalkyl,
(C₃-C₈)cycloalkyl(C₁-C₈)alkyl, (C₃-C₈)cycloalkyl(C₃-C₈)cycloalkyl, (C₁-C₈)alkylphenyl,
(C₁-C₈)alkoxy, aryl, cyano(C₁-C₈)alkyl, halo(C₁-C₈)alkyl having 1-9 identical or
different halogen atoms, (C₁-C₈)alkoxycarbonyl(C₁-C₈)alkyl, (C₁-C₈)alkoxy(C₁-C₈)alkyl
or halo(C₁-C₈)alkoxy(C₁-C₈)alkyl having 1-9 identical or different halogen atoms, and
Q¹ and Q² are mutually independently hydrogen, substituted (C₁-C₆)alkylsulphonyl, substituted alkoxycarbonyl(C₁-C₆)alkyl or substituted (C₁-C₆)haloalkyl sulphonyl,
comprising reacting one or more alcohols of formulae (IIa), (Ib) or (IIc) 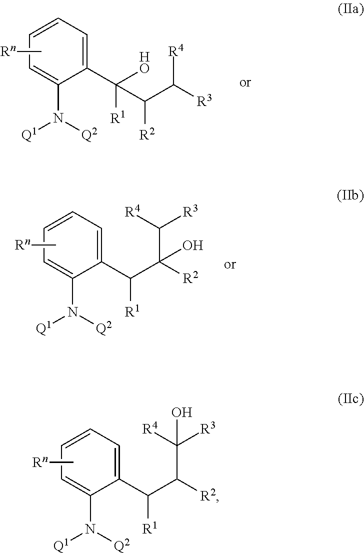
with one or more sulphonic acids.

2. Method according to claim 1, wherein
R is mutually independently halogen, (C₁-C₄)alkyl, (C₁-C₄)alkoxy, (C₁-C₄)alkylphenyl, aryl, cyano(C₁-C₄)alkyl, halo(C₁-C₄)alkyl having 1-9 identical or different halogen atoms, (C₁-C₄)alkoxycarbonyl(C₁-C₄)alkyl, (C₁-C₄)alkoxy(C₁-C₄)alkyl or halo(C₁-C₄)alkoxy(C₁-C₄)alkyl,
n is an integer from 0 to 3,
R¹, R², R³ and R⁴ are mutually independently hydrogen, (C₁-C₄)alkyl, (C₁-C₄)alkylphenyl, (C₁-C₄)alkoxy, aryl, cyano(C₁-C₄)alkyl, (C₁-C₄)alkoxycarbonyl(C₁-C₄)alkyl or (C₁-C₄)alkoxy(C₁-C₄)alkyl and
Q¹ and Q² are mutually independently hydrogen, substituted (C₁-C₄)alkylsulphonyl, substituted alkoxycarbonyl(C₁-C₄)alkyl or substituted (C₁-C₄)haloalkylsulphonyl.

3. Method according to claim 1, wherein
R is mutually independently fluorine, chlorine, bromine, methyl or trifluoromethyl,
n is an integer from 0 to 1,
R¹, R², R³ and R⁴ are mutually independently hydrogen, methyl, ethyl, n-propyl, isopropyl, n-butyl, isobutyl, sec-butyl or tert-butyl and
Q¹ and Q² are mutually independently hydrogen, substituted (C₁-C₄)alkylsulphonyl, substituted alkoxycarbonyl(C₁-C₃)alkyl or substituted (C₁-C₃)haloalkylsulphonyl.

4. Method according to claim 1, wherein
n is 0 or
R is fluorine and n is 1, wherein fluorine is optionally in the 5-, 6- or 7-position, optionally in the 6- or 7-position and optionally in the 7-position of the indane residue, or
R is trifluoromethyl and n is 1, wherein trifluoromethyl is optionally in the 5-, 6- or 7-position, optionally in the 6- or 7-position and optionally in the 7-position of the indane residue,
R¹, R², R³ and R⁴ are mutually independently hydrogen, methyl, ethyl, n-propyl, n-butyl, isobutyl or sec-butyl and
Q¹ and Q² are hydrogen.

5. Method according to claim 1, wherein the sulphonic acid used is methanesulphonic acid or trifluoromethanesulphonic acid.

6. Method according to claim 1, wherein the sulphonic acid used is trifluoromethanesulphonic acid.
